(19) United States
(12) Patent Application Publication
(10) Pub. No.: US 2004/0076972 A1
(43) Pub. Date: Apr. 22, 2004

(54) Marking

(76) Inventors: Jonathan Harold Wetton, Solihull (GB); Andrew John Hopwood, Solihull (GB)

Correspondence Address: Merchant & Gould, P O Box 2903, Minneapolis, MN, 55402-0903, US

(21) Appl. No.: 10/362,706

(22) PCT Filed: Sep. 3, 2001
(86) PCT No.: PCT/GB01/03929

(30) Foreign Application Priority Data
Sep. 1, 2000 (GB) 0021367.8

(51) Int. Cl. C12Q 1/68

(52) U.S. Cl. 435/6

(57) ABSTRACT

The invention provides a marking system, markers and methods of use of such marking systems and markers which enable unique marking of articles and subsequent detection of that marking. In particular the invention provides a marking system, the marking system comprising a plurality of different DNA fragment types, each of the plurality of different DNA fragment types comprising a plurality of different length DNA fragments and a method in which a sample of the DNA fragment type is taken, amplified and analysed to determine the identity of the marker for an article.

This invention concerns improvements in and relating to marking, particularly but not exclusively, to the marking, labelling or identification of items by the use of DNA.

A great variety of applications and situations make use of some form of marking of an item for security or other reasons. Some markings are intended to be visible, whilst a number of forms call for the marking to be invisible during normal use and only become visible in certain circumstances. Examples include inks which become visible under certain light conditions.

Certain other situations call for items to become marked in the event of certain circumstances arising and may additionally involve the transfer of the marking to individuals who come into contact with the marked item and/or to other locations which contact the item. Examples include the marking of bank notes with highly visible dye in the event of a robbery.

The present invention has amongst its aims to provide a marking system which is covert but can readily be inspected The present invention has amongst its aims the provision of a marking system which readily transfers and yet can be traced. The present invention has amongst its aims a marking system which can be readily examined using a minimum of investigating agents (such as primers) and/or investigation steps (such as sets of amplifications). The present invention has amongst its aims to provide a marking system which is easy to produce and use. The present invention has amongst its aims to provide a marking system which can readily provide a vast number of individual markers.

According to a first aspect of the present invention we provide a marking system, the marking system comprising a plurality of different DNA fragment types, each of the plurality of DNA fragment types comprising a plurality of different length DNA fragments.

Preferably the plurality of different DNA fragment types are included in a marker.

According to a second aspect of the invention we provide a marker, the marker including a DNA fragment type, the DNA fragment type including a plurality of different length DNA fragments.

The first and/or second aspect of the invention may include any of the features, options and possibilities set out in this document, including those which now follow.

Preferably the DNA fragment types differ from one another in terms of the specifics identity of two or three variables for each DNA fragment which form the DNA fragment types.

Preferably one of the variables is the length of the DNA fragments, particularly of a length variable portion of the DNA fragments. Preferably all DNA fragments within a DNA fragment type are different from each other.

Preferably one of the variables is the identity of a part of the sequence forming the DNA fragments, particularly the identity of one or more identity variable portions of the DNA fragments. Preferably the different identity variable portions are of the same length. Preferably the identity variable portion is or includes a portion towards the 5′ end of the DNA fragment. The identity variable portion may alternatively or additionally be a portion towards the 3′ end of the DNA fragment. Preferably the identity variable portion or portions are at the 5′ end and/or the 3′ end of the DNA fragment.

Different length DNA fragments in a DNA fragment type may have the same or different identities for a part of the sequence. More particularly, DNA fragments having different length length variable portions in a DNA fragment type may have the same or different identity variable portions.

Preferably the different identity variable portions are different due to a variation in one or more of the bases forming the DNA fragments, particularly variation in the DNA at the 5′ end of the DNA fragment.

In a preferred form of the invention the identity of the identity variable portion may vary due to differences in five or more bases of the sequence, more particularly in ten or more bases. Preferably the identity variable portions used and/or primers therefore will not hybridise with one another. It is preferred that a selected number of possible identities for the identity variable portions be provided for all the DNA fragments. The selected number may be three or four different possible identities. A selected number may be provided for each of the identity variable portions in the DNA fragments, particularly for the 5′ end and 3′ end identity variable portions. Preferably the selected number is the same for both ends of the DNA fragments. In a particularly preferred form the DNA fragments may have one of three possible identity variable portions at their 5′ end and one of three possible identity variable portions at their 3′ end. The DNA fragments may include one or more other portions, besides the one or more identity variable portions and length variable portion or portions.

In an alternative form of the invention the identity of the identity variable portion may only vary in terms of the first base at the 5′ end of a DNA fragment. DNA fragments with a G base or C base or T base or A base, particularly at the 5′ end, may be provided. Ideally, at least two such bases, more preferably three such bases and still more preferably four such bases are used to form different identity variable portions for DNA fragments. In such a form, preferably part of the variation which distinguishes between different DNA fragment types is provided as a part of the 5′ end portion of the DNA fragments. It is particularly preferred that the remainder of the 5′ portion of the DNA fragment be of 90% the same sequence and ideally completely the same sequence as the other DNA fragment 5′ end portions. In such a form, preferably the different DNA fragments are provided with a 3′ end portion and that 3′ end portion is at least 90% the same sequence, and ideally of completely the same sequence, for each of the different DNA fragments.

In a preferred form of the alternative case of the invention, three of four fragment types are provided, each fragment type providing thirteen different length DNA fragments. In that particularly preferred embodiment, the different DNA fragment types may be defined by the different identities of the 5′ end base identity. Preferably in the preferred embodiment, DNA fragments of the same length in each of the fragment types are of identical sequence to one another save for the 5′ end base variation.

Preferably the 5′ end identity variable portion and the 3′ end identity variable portion of the DNA fragments are connected by an intermediate sequence, preferably by the length variable portion. Preferably the plurality of different DNA fragments are provided by varying the length of the length variable portion between one DNA fragment and another DNA fragment in a DNA fragment type. Preferably at least five, more preferably at least six and ideally at least eight different DNA fragment lengths are provided in a DNA fragment type. The number of different DNA fragment lengths may be at least ten or even at least thirteen different lengths, provided by varying the length variable portion length.

Preferably DNA fragments of the same length are provided in each of the different DNA fragment types. Preferably all the DNA fragments in a DNA fragment type correspond in length with a DNA fragment in the other DNA fragment types. Preferably the same number of DNA fragments are provided in each of the DNA fragment types. Ideally the same number of fragments of the same lengths are provided in each of the DNA fragment types.

According to a third aspect of the invention we provide a method of marking an article, the method including provide a marking system, the marking system including a plurality of different DNA fragment types, each of the plurality of DNA fragment types including a plurality of different DNA fragments, the different DNA fragments being of different lengths, a known DNA fragment type being applied to the article.

According to a fourth aspect of the invention we provide a method of marking an article, the method including providing a known DNA fragment type, the DNA fragment type including a plurality of different length DNA fragments, the DNA fragment type being applied to the article.

The third and/or fourth aspect may include any of the features, options or possibilities set out elsewhere, particularly in the first and/or second aspects of the invention.

The DNA fragment type may be applied by contacting the DNA fragment type in liquid form with the article. The article may be wetted and/or soaked in the DNA fragment type. The DNA fragment type may be applied by painting or printing of the DNA fragment type on the article. The DNA fragment may be applied from solution to the article. The DNA fragment may be applied to the article as an aerosol.

The DNA fragment type may be applied to a part or the entirety of the article. The DNA fragment type may be applied to the external surface of the article and/or to an internal location of the article.

The DNA fragment type may be applied during the article's production, for instance during the formation of the article and/or during the finishing of the article and/or during the packaging of the article. The DNA fragment type may be applied to the article after production, for instance by the purchaser and/or on behalf of the purchaser.

According to a fifth aspect of the invention we provide a method of providing a potential marking for an article, the article being provided in proximity with a container, the method comprising provide a known DNA fragment type, the DNA fragment type including a plurality of different length DNA fragments, the DNA fragment type being applied to the article as a result of a disturbance to the container.

The fifth aspect of the invention may include any of the features, options or possibilities set out elsewhere, particularly in the first aspect of the invention.

The article may be provided in the container. The container may be a box, case or canister. The container may enclose the article against access and/or from view. The container may be openable, for instance using a key, security code or other activating device. In this way authorised access to the article(s) may be obtained and/or access to the article(s) may be obtained without disturbing the container.

The article(s) may be bank notes, cheques, vouchers or other paper or paper type goods having financial value. The article may be bank cards, credit cards, security cards or the like. A significant number of articles of the same or similar type may be provided within the container.

A disturbance to the container may include entry by unauthorised persons, entry at an unauthorised time or entry by unauthorised means. Disturbance to the container may include the breaking of the container or a part thereof, forced access to the container, damage to the container, the removal of the container from a location or a change in inclination to the container. Disturbance may comprise the removal of the container from a particular person or type of person's, such as security staff, possession.

The DNA fragment type may be applied to the article by the broaching of a barrier between the DNA fragment type and the article. The barrier may comprise an element separating a portion of the container containing the articles from the portion of the container containing the DNA fragment type and/or the breakage of a vessel containing the DNA fragment type and/or the breaking or removal of a portion thereof. The DNA fragment type may be provided within the container and/or attached thereto.

The DNA fragment type may be applied to the article by wetting of the article by the DNA fragment type. The DNA fragment type may flow and/or be sprayed and/or drop on to the article.

According to a sixth aspect of the present invention we provide a method of detecting the DNA marking of an article by a DNA fragment type from amongst a plurality of different DNA fragment types, each of the plurality of DNA fragment types comprising a plurality of different length DNA fragments, the method comprising obtaining a sample of DNA from the article, contacting the sample with an amplifying mixture, the amplifying mixture comprising two or more forward primers and one or more reverse primers, two or more of the primers providing detectable elements which are different from one another, amplifying the DNA fragment type and considering the identity of the detectable elements for at least some of the different length DNA fragments in the amplified fragment type.

The sixth aspect of the invention may include any of the features, options or possibilities set out elsewhere.

The sample of DNA may be obtained from the article by touching the article with an item, particularly a damp item, for instance a swab. The DNA may be removed from the item by washing. The DNA may be removed from the article by washing. The DNA may be recovered by centrifuging or filtration, particularly by centrifugal microfiltration.

The article may be solid or liquid. Examples of solid articles include paper goods, such as bank notes, cheques and other printed matter having or providing financial value. Examples of other articles include plastic goods; personal possessions such as jewellery, antiques and the like; precious goods such as paintings, antiques, furniture, jewellery and works of art; electronic goods, such as computers, computer peripheral devices, printers, microchips, disc drives and the like; goods requiring protection against counterfeiting such as clothing, watches, perfumes and the like.

The sample or one or more parts thereof may be amplified using PCR. Preferably the amplification process is performed using suitable primers for the DNA under consideration. A mixture of primers may be used to achieve amplification. A primer may be provided for each different identity variable portion that could be present. Preferably none of the primers anneal to variable identity portions other than their intended variable identity portion.

In a preferred case, three or four forward primers may be provided together with three or four, preferably a matching number, of reverse primers.

In an alternative case, preferably two, three or four forward primers and a single reverse primer are provided.

In this alternative case, the forward primers may have substantially identical (i.e. greater than 80 or greater than 90%) equivalent sequence to one another and are preferably identical with one another in sequence save for the 3′ end portion thereof. Ideally the only variation between forward primers is in the identity of the 3′ end base of the primers. Primers having an A or T or C or G 3′ end base may be provided. Preferably the forward primers are specific to one of the DNA fragment types due to the identity of the 3′ end variation used. Preferably a single reverse primer is provided. The reverse primer or primers preferably have a sequence which is at least 90% matching, ideally completely matching with the 3′ end of the DNA fragments, ideally of all the fragments.

The detectable elements provided on the forward primers are preferably different for each of the different forward primers used. The detectable elements provided on the reverse primers are preferably different for each of the different reverse primers used. The detectable elements may be dyes or other colour providing or generating means. The colour may be visible to the naked eye and/or to an analysis instrument. The colour may be immediately visible or require subsequent processing or action to render it visible. The detectable elements may be of other form, including radio emitters.

Preferably the method includes the amplification of all of the different length DNA fragments in the given DNA fragment type. The given DNA fragment type may preferably include eight or even thirteen different length DNA fragments. The DNA fragments of the given DNA fragment type may have six potential identity variable portions in the most preferred form of the invention. In an alternative case, three or four different identity variable portions may be present in any one of the fragments which make up a fragment type.

The identity of the detectable elements may be considered using a human eye, instrumentation for detecting colouration or instrumentation for detecting radio emissions or other characteristics of the detectable elements. The identity of the detectable elements may be considered by separating the different length DNA fragments from one another. Separation of the fragment lengths may be achieved by electrophoresis, for instance gel electrophoresis and/or capillary electrophoresis. Mass spectrometry may be used to determine the mass of the DNA fragment. The separation technique may provide a series of bands or locations, each band or location corresponding to a different length fragment. The detectable elements may indicate the particular primer or primers involved in the amplification of one or more of the different length fragments, preferably all. The bands or locations may indicate the identity of the identity variable portion or portions of the primer or primers involved in the amplification of the respective fragment for that band or area. The bands or locations may indicate the identity of the identity variable portion or portions of the DNA fragment and/or the length of the length variable portion of the DNA fragment.

Preferably all of the amplified fragments are considered in this manner. The results of the consideration may be expressed as the identity of the 3′ end identity variable portion and/or the identity of the 5′ end identity variable portion of the particular DNA fragment and/or a number representative of one or more of these variations.

The results may be compared with records or a database of marking systems to determine a match between the particular DNA fragment type of the sample and a known DNA fragment type and/or one or more recorded DNA fragment types. A match or a lack of a match may be used to confirm or deny the source of the article and/or the genuine nature of the article and/or contact of the article with an article marked with the DNA fragment type. The results may, therefore, be used to confirm physical contact between an article, such as a person, vehicle or the like with an article marked with the DNA fragment type, such as bank notes or the like, either directly or indirectly. The results may be used as evidence in the prosecution of a suspect.

Various aspects of the invention will now be described, by way of example only, and with reference to the accompanying drawings in which:

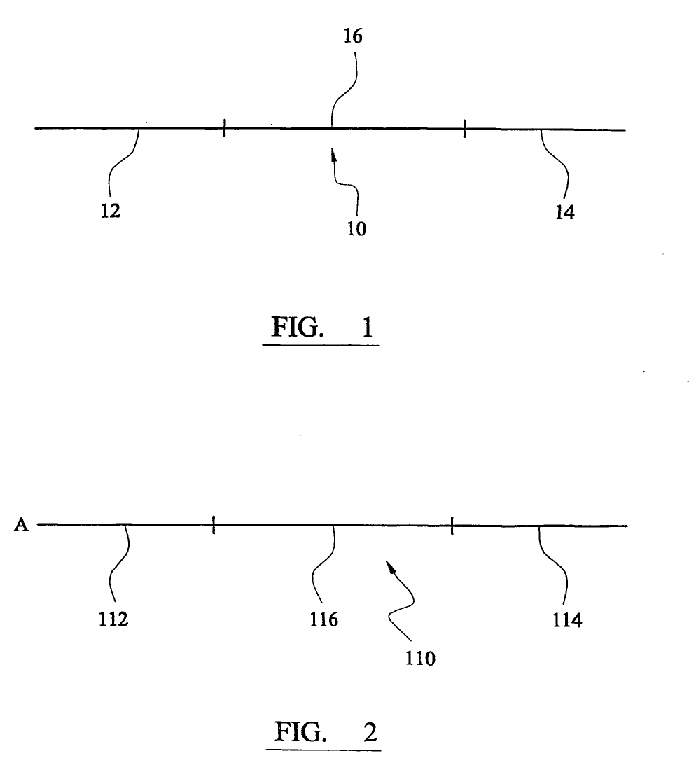

FIG. 1 illustrates schematically an example of a tag structure according to a preferred embodiment of the present invention; and

FIG. 2 illustrates schematically an example of a tag structure according to another embodiment of the present invention.

The invention aims to provide a marking system which is versatile and capable of use in a variety of situations, some of which are described in more detail below.

The general concept behind the invention is the provision of a distinct marker in each case where specific identification is required. The marker system is formed by makers having a very large number of DNA fragment type permutations. Each DNA fragment type being formed of a number of different sized DNA fragments, with further variation occurring in terms of one or both the 3′ and 5′ end sequences of each DNA fragment. A certain number different sized DNA fragments may be used in each DNA fragment type, with certain possible identities for the 3′ and/or 5′ end sequence. Thus a given DNA fragment will have a certain size (selected from the possible sizes used) and a certain 3′ and/or 5′ end sequence (selected from the possible sequences used). A significant number of different sizes and different 3′ and/or 5′ sequences soon leads to a very large number of possible permutations for the make-up of an individual DNA fragment type which is used to mark in a particular case.

By obtaining the variation through different sizes and carefully selected and provided variation in the 3′ and/or 5′ end sequences, however, the very large number of permutations is achieved whilst still allowing a quite limited number of primers to effect the analysis process. This means that the cost of providing the primers and the time and cost involved in performing the analysis is kept low. This contrast with a potential system which could be based around a very large number of different and unrelated DNA sequences to make up the marker system.

Preferred Embodiment of Invention

In the preferred embodiment of the invention the above mentioned general concept is deployed. The general form of a DNA fragment, with a number of such DNA fragments making up a DNA fragment type, is illustrated in FIG. 1.

The overall DNA fragment 10 includes a 5′ end portion, 12, a 3′ end portion 14, and an intermediate portion 16. The intermediate portion 16 is the part of the fragment 10 in which the variation to achieve DNA fragments 10 of different length is provided. The number of bases in the intermediate portion 16 in one fragment 10 is thus different from the number in another fragment 10 and the other fragments 10 which go to make up the size variation in any given DNA fragment type of the marker system.

In this particular embodiment of the invention eight different sizes are used in the intermediate portion 16 to give eight different sized DNA fragments in each DNA fragment type.

As well as the variation in size of a DNA fragment, the embodiment also provides a number of different sequences for the 5′ end portion 12 and the 3′ end portion 14. In the particular embodiment of the invention the 5′ end portion 12 will have one of three designed sequences and the 3′ end portion 14 will also have one of three, different, designed sequences. Thus a given DNA fragment 10 will have a particular size (of eight options), particular 5′ end portion (of three options) and a particular 3′ end portion (of three options).

The different 5′ end portions and 3′ end portion sequences are designed so that they can be effectively amplified using a multiplex of primers, with comparable optimum amplification conditions and matching efficiency to one another.

Within a DNA fragment type, eight different DNA fragments 10 are provided, each with its own unique size (relative to the other DNA fragments 10 in the DNA fragment type) and each of the DNA fragments will have one of the three 5′ end portion sequences and one of the three 3′ end portion sequences.

The fragment type can be deployed in a marker in a number of ways, some of which are exemplified below.

The marker from a marking system may be a applied to an article in the event of certain circumstances arising and will then remain on the article during its subsequent life, or at least at a significant time period. The circumstances may be the disturbance of the article and/or a container for the article. In one example the marker may be provided in a container within the case for an amount of money as a security device. In the event of the case being broken into the container is designed to break and hence bring the marker into contact with the money. Any subsequent contact of the money with persons, items or locations is designed to give partial transfer of the marker to those articles. The marker is thus intended to allow the money stolen, persons handling that money and cars, houses and the like which are linked to the robbery.

The marker can be applied to an article during a stage of that articles production and remain a feature of it during its subsequent life or be added by the purchaser themselves at a later date. In this form the marker can be used to verify the genuine nature of the article, for instance genuine rather than counterfeit perfume, and/or to identify a feature of the articles production, for instance the particular location of the producer which made the goods so as to trace the source of production should a problem arise. The markers of such a marking system enables these benefits, but without interfering with the articles normal use or appearance. In this form it is desirable for the DNA to be retained by the article in the event of contact with another article.

When an article needs to have its DNA fragment mixture decoded to investigate the source of that article, for instance, a sample of the DNA is recovered. This may involve swabbing the article with a damp cotton swab. The lifted sample of the DNA fragment type marker is then subjected to washing and centrifugal micro filtration to obtain the sample for subsequent analysis. As an alternative, where the article is suited, an area of the article bearing the marker, or even the whole article, may be washed (sterile water or buffered solution) to remove the DNA fragment type with the sample subsequently being purified using centrifugal micro filtration.

Once obtained, the sample of the DNA fragment type is contacted with a mixture of primers, the mixture including a forward primer for each of the possible 5′ end portion sequences in the design and a reverse primer for each of the possible 3′ end portion sequences of the design. As stated above, in this particular example there are three possible 5′ end portion and three possible 3′ end portions and hence three forward primers and three reverse primers are provided, each of these oligonucleotides being specific to one of the six sequences of the end portions.

As only one of the three forward primers will match the 5′ end portion sequence and as only one of the three reverse primers will match reverse sequence pairing to the 3′ end portion sequence, only those two primers will anneal and hence only they will amplify that DNA fragment. An equivalent procedure applies to each of the other different size DNA fragments which make up the DNA fragment type under analysis, the particular forward primer and particularly reverse primer which anneals in each case varying according to the variations in the 5′ end portion and 3′ end portion between DNA fragments.

As each of the forward and reverse primers not only varies in terms of its sequence but also varies in terms of its labelling the primers which actually anneal at the forward 5′ end and reverse 5′ end can both be identified. The preferred embodiment of the invention uses a different coloured dye label for each of the forward primers and each of the reverse primers. The same three colours can be used for each or different colours can be used.

The overall result is that the amplification products are labelled with a colour which is specific to the identity of the forward primer and reverse primer in that case and hence specific to the 5′ end portion and 3′ end portion in the DNA fragment types used as the particular marker selected from the marking system for that particular article.

Once the amplification process has been completed, which is easy to operate due to the matching conditions needed for the limited number of primers needed, even allowing for the very great number of permutations which are accommodated, the amplification products can be separated and then inspected.

In one embodiment the inspection process uses gel electrophoresis to separate the amplified fragment lengths according to their length/size. The results can then be considered to determine the colour or colours of the labels which have become associated with each DNA fragment in the DNA fragment type by virtue of the forward and reverse primers which annealed. For instance as summarised in Table 1 with three forward primers and three reverse primers used to investigate a DNA fragment type formed of eight different size DNA fragments with three potential 5′ end portions and three potential 3′ end portions, the following results might be obtained:

TABLE 1

| Detected | | | |
|---|---|---|---|
| colours from | | | |
| lowest to | | | |
| highest | | | |
| molecular | Indicated | Indicated | Code Number |
| weight of | Forward | Reverse | Representing |
| product. | Primer | Primer | Result |
| Yellow/Yellow | 1 | 1 | 1 |
| Blue/Green | 2 | 3 | 4 |
| Blue/Blue | 2 | 2 | 5 |
| Yellow/Blue | 1 | 2 | 2 |
| Green/Green | 3 | 3 | 6 |
| Yellow/Green | 1 | 3 | 3 |
| Green/Green | 3 | 3 | 6 |
| Blue/Green | 2 | 3 | 4 |

The colours, end portions and particularly the representative number can be used to compare the DNA obtained with records, for instance to link the sample with a particular tagged article.

Increasing the number of different fragment lengths and/or increasing the number of end portion sequences increases the number of combinations which are possible.

Alternative Embodiment of Invention

The alternative embodiment of the system is based around the same underlying concept but differs in the exact manner in which the variations are provided.

This embodiment once again involves the use of one of a number of DNA fragment types to mark an item. Each DNA fragment type includes a series of DNA fragments which are different from one another due to their being of a variety of sizes. Each of the DNA fragments is based on the form illustrated in FIG. 2.

Each fragment 110 is formed of a forward universal sequence portion 112 and a reverse universal sequence 114, the two being separated by a variable length portion 116. By using the various lengths of the sequence forming the variable length portion 116 which are possible, whilst using the same forward universal sequence portion 112 and reverse universal portion 114, the different lengths are achieved.

In this embodiment of the invention 13 different length fragments are used in each fragment type.

Variation between the different fragment types is achieved by varying the identity of the initial base in the forward universal sequence portion 112. Thus one fragment type may be provided with a T base at this location 18 of the fragment 10, whereas the other fragment types may be provided with a C base or G base or A base.

The net result is that up to 4 different ends are provided for each of a series of 13 different length fragments which form a DNA fragment type.

In this particular embodiment three different ends are considered. By selecting one of the three possible ends for each of the 13 different lengths a DNA fragment type is produced, the fragment type being one of greater than 1.59 million possible types.

By selecting one of the four possible ends for each of the 13 different lengths a fragment type is once again produced, the fragment type being one of greater than 67 million possible types.

When an article bearing a marker having a particular DNA fragment type needs to be analysed, a sample of the DNA fragment type can be obtained in the manner outlined above for the preferred embodiment of the invention.

Once the sample has been obtained, the DNA sample is then contacted with a number of forward primers corresponding to the number of possible different end bases and a reverse primer.

As the reverse universal sequence is common to all the fragment types and all the fragment lengths it will achieve the necessary reverse extension of the amplification process.

The forward primers used will depend on the number of different ends which may potentially have been used. Generally four forward primers will be used with each of the primers having a common sequence apart from the last base, the sequence also being common with the sequence which matches the forward universal portion 12. The last bases of the four primers are, however, different from one another, T, A, G, C and as a consequence mean that only one forward primer will anneal and subsequently amplify a fragment length. The T starting primer will amplify an A starting fragment length, an A starting primer for a T starting fragment length, a C staring primer for a G staring fragment length and a G starting primer for a C starting fragment length. As a result of the four primers added amplification of each of the fragment lengths is achieved, with the primers varying from length to length depending on the identity of the starting base.

If the fragment mixture is known to include only 3 starting base variations then only 3 primers would be needed.

Each of the primers not only varies in terms of the starting base identity but also varies in terms of its colour labelling, a different colour being used for each primer. The result is that the amplification products are labelled with a colour which is specific to the starting base of the forward primer and specific to the starting base of the fragment length.

Once the amplification process has been completed, it can be separated into different sizes and analysed in an equivalent manner to that described above for the preferred embodiment of the invention. By considering the results it is possible to determine the colour of the label which has become associated with each fragment length and as a consequence the primer start and fragment start. Results of this type are summarised in Table 1 with three primers used, to form a representative number.

TABLE 1

| Detected | | | |
|---|---|---|---|
| colours from | | | |
| lowest to | | | |
| highest | | Indicated | |
| molecular | Indicated | fragment | Code number |
| weight of | Primer | length | representing |
| product. | Starting base | starting base | result |
| Blue | T | A | 1 |
| Blue | T | A | 1 |
| Green | A | T | 2 |
| Yellow | G | C | 3 |
| Blue | T | A | 1 |
| Green | A | T | 2 |
| Blue | T | A | 1 |
| Yellow | G | C | 3 |
| Yellow | G | C | 3 |
| Green | A | T | 2 |
| Yellow | G | C | 3 |
| Green | A | T | 2 |
| Blue | T | A | 1 |

The colours, base identities and particularly the representative number can be used to compare the DNA obtained with records, for instance to link the sample with a particular tagged article.

Increasing the number of different lengths increases the number of combinations which are possible, just as for the other embodiment of the invention.

1. A marking system, the marking system comprising a plurality of different DNA fragment types, each of the plurality of DNA fragment types comprising a plurality of different DNA fragments, all the DNA fragments within a DNA fragment type being different from one another, each of the different DNA fragments within a DNA fragment type being different in length to one another due to the length variable portion being different, each DNA fragment further including one or two identity variable portions, amplification of the DNA fragment type providing detectable elements, the identity of the one or two identity variable portions determining the identity of the detectable elements provided by amplification.

2. A marking system according to claim 1 in which the different identity variable portions are of the same length.

3. A marking system according to claim 1 or claim 2 in which the identity variable portions at the 5′ end and the 3′ end of the DNA fragment are provided.

4. A marking system according to any preceding claim in which the invention the identity of the identity variable portion varies due to differences in five or more bases of the sequence.

5. A marking system according to any preceding claim in which the identity variable portions used and/or primers therefore will not hybridise with one another.

6. A marking system according to any preceding claim in which a selected number of possible identities for the identity variable portions is provided for all the DNA fragments.

7. A marking system according to any preceding claim in which a selected number of possible identities for the identity variable portions is provided for each of the identity variable portions in the DNA fragments, particularly for the 5′ end and 3′ end identity variable portions.

8. A marking system according to any preceding claim in which the DNA fragments have one of three possible identity variable portions at their 5′ end and one of three possible identity variable portions at their 3′ end.

9. A marking system according to any of claims 1 to 3 in which the identity of the identity variable portion only varies in terms of the first base at the 5′ end of a DNA fragment.

10. A marking system according to any preceding claim in which a 5′ end identity variable portion and a 3′ end identity variable portion of the DNA fragments are connected by the length variable portion.

11. A marking system according to any preceding claim in which at least five different DNA fragment lengths are provided in a DNA fragment type.

12. A marking system according to any preceding claim in which all the DNA fragments in a DNA fragment type each correspond in length with a DNA fragment in the other DNA fragment types.

13. A marker, the marker including a DNA fragment type, the DNA fragment type including a plurality of different DNA fragments, all the DNA fragments within a DNA fragment type being different from one another, each of the different DNA fragments within a DNA fragment type being different in length to one another due to the length variable portion being different, each DNA fragment further including one or two identity variable portions, amplification of the DNA fragment type providing detectable elements, the identity of the one or two identity variable portions determining the identity of the detectable elements provided by amplification.

14. A marker according to claim 13 provided with any of the DNA fragment and/or DNA fragment types of claims 1 to 12.

15. A method of marking an article, the method including provide a marking system, the marking system comprising a plurality of different DNA fragment types, each of the plurality of DNA fragment types comprising a plurality of different DNA fragments, all the DNA fragments within a DNA fragment type being different from one another, each of the different DNA fragments within a DNA fragment type being different in length to one another due to the length variable portion being different, each DNA fragment further including one or two identity variable portions, amplification of the DNA fragment type providing detectable elements, the identity of the one or two identity variable portions determining the identity of the detectable elements provided by amplification, a known DNA fragment type being applied to the article.

16. A method according to claim 15 in which the DNA fragment type is applied by contacting the DNA fragment type in liquid form with the article.

17. A method according to claim 15 or claim 16 in which the article is wetted and/or soaked in the DNA fragment type and/or the DNA fragment type is applied by painting or printing of the DNA fragment type on the article and/or the DNA fragment is applied from solution to the article and/or the DNA fragment may be applied to the article as an aerosol.

18. A method according to any of claims 15 to 17 in which the DNA fragment type is applied to a part of the article and/or the DNA fragment type is applied to the external surface of the article and/or to an internal location of the article.

19. A method according to any of claims 15 to 18 in which the DNA fragment type is applied during the article's production, and/or during the finishing of the article and/or during the packaging of the article and/or after production.

20. A method of providing a potential marking for an article, the article being provided in proximity with a container, the method comprising provide a known DNA fragment type, the known DNA fragment type comprising a plurality of different DNA fragments, all the DNA fragments within the known DNA fragment type being different from one another, each of the different DNA fragments within the known DNA fragment type being different in length to one another due to the length variable portion being different, each DNA fragment further including one or two identity variable portions, amplification of the DNA fragment type providing detectable elements, the identity of the one or two identity variable portions determining the identity of the detectable elements provided by amplification, the DNA fragment type being applied to the article as a result of a disturbance to the container.

21. A method according to claim 20 in which the container encloses the article against access and/or from view.

22. A method according to claim 20 or claim 21 in which authorised access to the article(s) is obtained without disturbing the container.

23. A method according to any of claims 20 to 22 in which the article(s) are bank notes cheques, vouchers or other paper or paper type goods having financial value, bank cards, credit cards or security cards.

24. A method according to any of claims 20 to 23 in which a disturbance to the container includes entry by unauthorised persons, entry at an unauthorised time or entry by unauthorised means or the breaking of the container or a part thereof or forced access to the container or damage to the container or the removal of the container from a location or a change in inclination to the container or the removal of the container from a particular person or type of person's possession.

25. A method according to any of claims 20 to 24 in which the DNA fragment type is applied to the article by the broaching of a barrier between the DNA fragment type and the article.

26. A method according to any of claims 20 to 25 in which the DNA fragment type is applied to the article by wetting of the article by the DNA fragment type.

27. A method of detecting the DNA marking of an article by a DNA fragment type from amongst a plurality of different DNA fragment types, each of the plurality of DNA fragment types comprising a plurality of different DNA fragments, all the DNA fragments within a DNA fragment type being different from one another, each of the different DNA fragments within a DNA fragment type being different in length to one another due to the length variable portion being different, each DNA fragment further including one or two identity variable portions, amplification of the DNA fragment type providing detectable elements, the identity of the one or two identity variable portions determining the identity of the detectable elements provided by amplification, the method comprising obtaining a sample of DNA from the article, contacting the sample with an amplifying mixture, the amplifying mixture comprising two or more forward primers and one or more reverse primers, two or more of the primers providing detectable elements which are different from one another, amplifying the DNA fragment type and considering the identity of the detectable elements for at least some of the different length DNA fragments in the amplified fragment type.

28. A method according to claim 27 in which a primer is provided for each different identity variable portion that could be present.

29. A method according to claim 27 or 28 in which three or four forward primers are provided together with a matching number of reverse primers.

30. A method according to claim 28 or claim 20 in which four forward primers and a single reverse primer are provided.

31. A method according to any of claims 28 to 30 in which the detectable elements provided on the forward primers and/or the reverse primer are different for each of the different forward primers used.

32. A method according to any of claims 28 to 31 in which the detectable elements are dyes or other colour providing or generating means.

33. A method according to any of claims 28 to 32 in which the identity of the detectable elements is considered by separating the different length DNA fragments from one another.

34. A method according to any of claims 28 to 33 in which the detectable elements indicate the particular primer or primers involved in the amplification of one or more of the different length fragments.

35. A method according to any of claims 28 to 34 in which the bands or locations indicate the identity of the identity variable portion or portions of the DNA fragment and/or the length of the length variable portion of the DNA fragment.

36. A method according to any of claims 28 to 35 in which the results are compared with records or a database of marking systems to determine a match between the particular DNA fragment type of the sample and a known DNA fragment type and/or one or more recorded DNA fragment types.

37. A method according to any of claims 23 to 36 in which a match or a lack of a match is used to confirm or deny the source of the article and/or the genuine nature of the article and/or contact of the article with an article marked with the DNA fragment type.
